(19) United States
(12) Patent Application Publication
(10) Pub. No.: US 2023/0136130 A1
(43) Pub. Date: May 4, 2023

(54) HEMATOCRIT SENSOR BASED ON IMPEDANCE MEASUREMENT IN CARDIOVASCULAR IMPLANTABLE ELECTRONIC DEVICES

(71) Applicant: Medtronic, Inc., Minneapolis, MN (US)

(72) Inventor: Geert Morren, Vissenaken (BE)

(21) Appl. No.: 17/955,607

(22) Filed: Sep. 29, 2022

Related U.S. Application Data
(60) Provisional application No. 63/274,295, filed on Nov. 1, 2021.

(51) Int. Cl. A61B 5/0538; A61B 5/02; A61B 5/145; A61B 5/00

(52) U.S. Cl. CPC A61B 5/0538; A61B 5/02028; A61B 5/14535; A61B 5/4875; A61B 2560/0223

(57) ABSTRACT

An example device includes memory configured to store a first impedance and a second impedance each associated with a heart of a patient, and processing circuitry communicatively coupled to the memory. The processing circuitry is configured to determine at least one of a measure of hematocrit, an indication of fluid overload, or an indication of true anemia based at least in part on the first impedance and the second impedance. The processing circuitry is configured to output an indication of the measure of hematocrit, the indication of fluid overload, or the indication true anemia.

### RELATED APPLICATIONS

This application claims the benefit of U.S. Provisional Application Ser. No. 63/274,295, filed August Nov. 1, 2021, the entire contents of each of which are incorporated herein by reference.

### TECHNICAL FIELD

The disclosure relates to medical devices, and, more particularly, to cardiovascular medical device systems configured to sense hematocrit in a patient.

### BACKGROUND

Implantable cardioverter defibrillators (ICDs) and implantable artificial pacemakers may provide cardiac pacing therapy to a patient's heart when the natural pacemaker and/or conduction system of the heart fails to provide synchronized atrial and ventricular contractions at rates and intervals sufficient to sustain healthy patient function. Such antibradycardial pacing may provide relief from symptoms, or even life support, for a patient. Cardiac pacing may also provide electrical overdrive stimulation, e.g., ATP therapy, to suppress or convert tachyarrhythmias, again supplying relief from symptoms and preventing or terminating arrhythmias that could lead to sudden cardiac death. Cardiac resynchronization therapy (CRT) is another type of cardiac pacing that may help enhance cardiac output by resynchronizing the electromechanical activity of the ventricles of the heart. Ventricular desynchrony may occur in patients that suffer from heart failure.

Treatment of end stage heart failure may include implant of a mechanical circulatory support device (e.g., a ventricular assist device, such as a left ventricular assist device) to aid the heart in pumping blood to the body. A ventricular assist device may be used to sustain life until a heart transplant procedure may be performed (e.g., as a bridge to transplant), as a permanent solution to reduce the symptoms of heart disease (e.g., destination therapy), or as a temporary measure to treat a reversible condition (such as, e.g., myocarditis). Though ventricular assist devices may be effective in the treatment or management of symptoms of heart failure, ventricular assist patients may experience an increased risk of a stroke.

Additionally, some medical devices, such as an insertable cardiac monitor or pressure monitor, may monitor physiological parameters of a patient. For example, some medical devices are configured to sense cardiac electrogram (EGM) signals, e.g., electrocardiogram (ECG) signals, indicative of the electrical activity of the heart via electrodes.

### SUMMARY

Hematocrit is a relatively important parameter for managing patients with heart failure and other chronic diseases. In combination with the existing diagnostics in cardiac implantable electronic devices (such as intrathoracic impedance), a hematocrit sensor can improve remote patient management. For example, a hematocrit sensor may be used to detect anemia in a patient, which is a common comorbidity of heart failure.

Hematocrit of blood samples (in-vitro, in a fixed geometry) may be determined by sensing the conductance of blood because the conductivity of blood is inversely correlated with the hematocrit. When the impedance is measured intracardially (in-vivo), however, there are many confounding factors, such as changes in the sample volume/geometry and motion.

30% to 50% of congestive heart failure (CHF) patients have anemia, which may be defined as low hemoglobin or low hematocrit values, and the prevalence of anemia generally increases with disease severity. Anemia is an independent risk factor for a number of adverse outcomes. Routine detection of and management of anemia in patients with chronic conditions such as CHF may be desirable. A reduced hemoglobin/hematocrit can result from either a reduced red blood cell (RBC) volume (which may be referred to herein as “true anemia”) or from an increased plasma volume and fluid overload. Both causes of anemia (true anemia and fluid overload) may require different treatments. For example, treatment for true anemia may depend on pathophysiology, for example, intravenous administration of iron in the case of iron deficiency. Treatment for fluid overload may include, for example, the administration of diuretics to the patient. Therefore, there may be a need to, relatively quickly, distinguish between true anemia and fluid overload in patients and provide an indication of which condition a patient may be suffering from, thereby facilitating the administration by a clinician of the appropriate treatment for the condition of the patient. A device capable of detecting anemia, and differentiating true anemia from fluid overload, may improve patient management by enabling earlier detection and treatment of the underlying cause of the anemia.

In one example, this disclosure is directed to a method including: determining, by processing circuitry, a first impedance associated with a heart of a patient; determining, by the processing circuitry, a second impedance associated with the heart of the patient; determining, by the processing circuitry, at least one of a measure of hematocrit, an indication of fluid overload, or an indication of true anemia based at least in part on the first impedance and the second impedance; and outputting, by the processing circuitry, an indication of the measure of hematocrit, the indication of fluid overload, or the indication true anemia.

In another example, this disclosure is directed to a device including: memory configured to store a first impedance and a second impedance, each associated with a heart of a patient; and processing circuitry communicatively coupled to the memory and configured to: determine at least one of a measure of hematocrit, an indication of fluid overload, or an indication of true anemia based at least in part on the first impedance and the second impedance; and output an indication of the measure of hematocrit, the indication of fluid overload, or the indication true anemia.

In a further example, this disclosure is directed to a non-transitory computer-readable storage medium having stored thereon instructions which, when executed, cause processing circuitry to: determine at least one of a measure of hematocrit, an indication of fluid overload, or an indication of true anemia based at least in part on a first impedance and a second impedance associated with a heart of a patient; and output an indication of the measure of hematocrit, the indication of fluid overload, or the indication true anemia.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is a conceptual diagram illustrating an example implantable medical device system.

FIG. 2 is a block diagram illustrating an example configuration of an external device of FIG. 1.

FIG. 3 is a block diagram illustrating an example configuration of a pacemaker/cardioverter/defibrillator of the implantable medical device system of FIG. 1.

FIG. 4 is a graphical diagram illustrating an example phase shift between in input current and an output voltage.

FIG. 5A is a graphical diagram illustrating an example of conductivity of heart and blood tissue as a function of frequency.

FIG. 5B is a graphical diagram illustrating an example of permittivity of heart and blood tissue as a function of frequency.

FIG. 6 is a chart illustrating an example relationship between impedance measurements which may be used to discriminate between true anemia and fluid overload according to the techniques of this disclosure.

FIG. 7A is a conceptual diagram illustrating an example positioning of leads and electrodes used to sense an intrathoracic impedance according to the techniques of this disclosure.

FIG. 7B is a conceptual diagram illustrating an example positioning of leads and electrodes used to sense an intracardiac impedance according to the techniques of this disclosure.

FIGS. 8A and 8B are conceptual diagrams illustrating example electrode locations.

FIG. 9 is a flow diagram illustrating example hematocrit sensing techniques of this disclosure.

### DETAILED DESCRIPTION

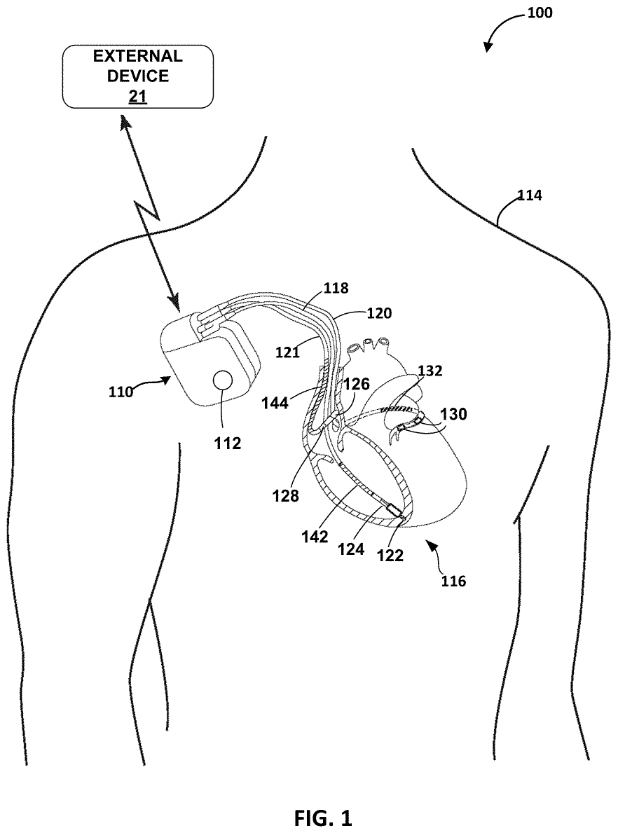

FIG. 1 is a schematic diagram of an example implantable medical device system 100 in conjunction with a patient 114. As illustrated in FIG. 1, a medical device system 100 for sensing cardiac events (e.g., P-waves, R-waves, QRS complexes, etc.) and detecting tachyarrhythmia episodes may include an implantable medical device (IMD) 110, right ventricular lead 118, left ventricular lead 120, and atrial lead 121. In one example, IMD 110 may be embodied as an implantable cardioverter-defibrillator (ICD) or pacemaker/cardioverter/defibrillator (PCD) capable of delivering pacing, cardioversion and defibrillation therapy to the heart 116 of a patient 114. Right ventricular lead 118, left ventricular lead 120 and atrial lead 121 are electrically coupled to PCD 110 and extend into the patient's heart 116 via a vein. Right ventricular lead 118 includes electrodes 122 and 124 shown positioned on the lead in the patient's right ventricle (RV) for sensing ventricular electrogram signals and pacing in the RV. Left ventricular lead 120 includes one or more electrodes 130 positioned in the patient's left ventricle (LV) for sensing ventricular electrogram signals and pacing the LV. Atrial lead 121 includes electrodes 126 and 128 positioned on the lead in the patient's right atrium (RA) for sensing atrial electrogram signals and pacing in the RA.

In the illustrated example, each of right ventricular lead 118, left ventricular lead 120, and atrial lead 121 may carry a high voltage coil electrode 142, 132, and 144, respectively, used to deliver cardioversion and defibrillation shocks. In other examples, system 100 may include any number of high voltage coil electrodes on any one or more leads. In some examples, right ventricular lead 118 may include two high voltage coil electrodes, e.g., a first high voltage coil electrode in the RV and a second high volage coil electrode in the RA or superior vena cava (SVC). The techniques disclosed herein may be applicable to systems including any number of electrodes and/or leads, including systems that do not include high voltage coil electrodes and IMDs that do not deliver antitachyarrhythmia shocks.

Implantable medical device circuitry configured for performing the functions of PCD 110 described herein and associated battery or batteries are housed within a sealed housing 112. Housing 112 may be conductive so as to serve as an electrode for use as an indifferent electrode during pacing or sensing or as an active electrode during defibrillation. As such, housing 112 is also referred to herein as “housing electrode” 112. Each of left ventricular lead 120, right ventricular lead 118, and atrial lead 121 may be used to acquire intracardiac electrogram signals and hematocrit measurements from the patient 114 and/or to deliver therapy in response to the acquired data. PCD 110 may utilize two or more electrodes on the leads, or one or more electrodes on the leads in combination with housing electrode 112, for sensing and therapy.

EGM signal data, hematocrit data, and/or true anemia or fluid overload data acquired by PCD 110 may be transmitted to an external device 21. External device 21 may be embodied as a programmer, e.g., used in a clinic or hospital to communicate with PCD 110 via wireless telemetry. External device 21 may be coupled to a remote patient monitoring system, such as Carelink™, available from Medtronic plc, of Dublin, Ireland. External device 21 is used to program commands or operating parameters into PCD 110 for controlling PCD function and to interrogate PCD 110 to retrieve data, including device operational data as well as physiological data accumulated in a memory of PCD 110. Examples of communication techniques used by PCD 110 and external device 21 include low frequency or radiofrequency (RF) telemetry, which may be an RF link established via Bluetooth®, WiFi, medical information communication service (MICS), or another public or proprietary communication protocol.

External device 21 may be configured to communicate with PCD 110. In some examples, external device 21 comprises a handheld computing device, computer workstation, or networked computing device. External device 21 may include a user interface that receives input from a user. In other examples, the user may also interact with external device 21 remotely via a networked computing device. The user may interact with external device 21 to communicate with PCD 110. For example, the user may interact with external device 21 to send an interrogation request and retrieve therapy delivery data, update therapy parameters that define therapy, or perform any other activities with respect to PCD 110. In some examples, external device 21 may receive from PCD 110 an indication of a measurement of hematocrit, an indication of true anemia, or an indication of fluid overload, which a clinician may use to guide treatment of patient 114. Although the user is a physician, technician, surgeon, electrophysiologist, or other healthcare professional, the user may be patient 14 in some examples.

External device 21 may also allow the user to define how PCD 110 senses electrical signals (e.g., ECGs), detects arrhythmias such as tachyarrhythmias, detects hematocrit, detects true anemia, detects fluid overload, delivers therapy, and communicates with other devices of system 100. For example, external device 21 may be used to change tachyarrhythmia detection parameters. In another example, external device 21 may be used to manage therapy parameters that define therapies such as CRT or ATP therapy.

External device 21 may communicate with PCD 110 via wireless communication using any techniques known in the art. Examples of communication techniques are described above with reference to FIG. 1. In some examples, external device 21 may include a programming head that may be placed proximate to patient 14's body near the PCD 110 implant site in order to improve the quality or security of communication between PCD 110 and external device 21.

The example illustrated in FIG. 1 should not be considered limiting of the techniques described herein. The techniques disclosed herein may be applicable to systems that do not include any leads and/or that do not provide any electrical stimulation therapies, such as insertable/implantable cardiac monitors, implantable pressure sensors, or other implantable systems.

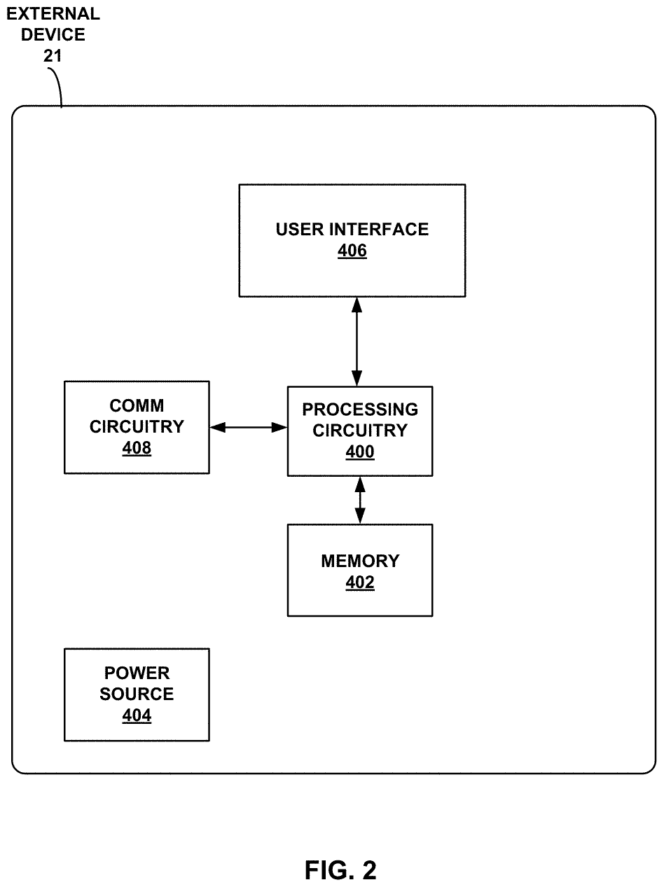

FIG. 2 is a functional block diagram illustrating an example configuration of external device 21. External device 21 may include processing circuitry 400, memory 402, communication circuitry 408, user interface 406, and power source 404. Processing circuitry 400 controls user interface 406 and communication circuitry 408 and stores and retrieves information and instructions to and from memory 402. External device 21 may be configured for use as a clinician programmer or a patient programmer. Processing circuitry 400 may comprise any combination of one or more processors including one or more microprocessors, DSPs, ASICs, FPGAs, or other equivalent integrated or discrete logic circuitry. Accordingly, processing circuitry 400 may include any suitable structure, whether in hardware, software, firmware, or any combination thereof, to perform the functions ascribed herein to processing circuitry 400.

A user, such as a clinician or patient 114, may interact with external device 21 through user interface 406. User interface 406 may include a display, such as an LCD or LED display or other type of screen, to present information. In some examples, user interface 406 may be configured to alert a clinician of a measure of hematocrit, an indication of true anemia, or an indication of fluid overload of patient 114, such that the clinician may take the appropriate measures to treat patient 114. In addition, user interface 406 may include an input mechanism to receive input from the user. The input mechanisms may include, for example, buttons, a keypad (e.g., an alphanumeric keypad), a peripheral pointing device or another input mechanism that allows the user to navigate through user interfaces presented by processing circuitry 400 of external device 21 and provide input.

In some examples, at least some of the techniques described herein may be implemented by processing circuitry 400 of external device 21. For example, processing circuitry 400 may determine hematocrit values based on impedance values measured by PCD 110, and/or may determine whether to indicate fluid overload or anemia of patient 114 based on data received from PCD 110.

Memory 402 may include instructions for operating user interface 406 and communication circuitry 408, and for managing power source 404. Memory 402 may also store any data retrieved from PCD 110. The clinician may use this therapy data to determine the progression of the patient condition in order to determine future treatment. Memory 402 may include any volatile or nonvolatile memory, such as RAM, ROM, EEPROM or flash memory. Memory 402 may also include a removable memory portion that may be used to provide memory updates or increases in memory capacities. A removable memory may also allow sensitive patient data to be removed before external device 21 is used by a different patient.

Wireless telemetry in external device 21 may be accomplished by use of communication circuitry 408, which may communicate with a proprietary protocol or industry-standard protocol such as using the Bluetooth® specification set. Accordingly, communication circuitry 408 may be similar to the communication circuitry contained within by PCD 110. In alternative examples, external device 21 may be capable of infrared communication or direct communication through a wired connection. In this manner, other external devices may be capable of communicating with external device 21 without needing to establish a secure wireless connection.

Power source 404 may deliver operating power to the components of external device 21. Power source 404 may include a battery and a power generation circuit to produce the operating power. In some examples, the battery may be rechargeable to allow extended operation. Recharging may be accomplished by electrically coupling power source 308 to a cradle or plug that is connected to an alternating current (AC) outlet. In addition, recharging may be accomplished through proximal inductive interaction between an external charger and an inductive charging coil within external device 21. In other examples, traditional batteries (e.g., nickel cadmium or lithium ion batteries) may be used. In addition, external device 21 may be directly coupled to an alternating current outlet to operate. Power source 404 may include circuitry to monitor power remaining within a battery. In this manner, user interface 406 may provide a current battery level indicator or low battery level indicator when the battery needs to be replaced or recharged. In some cases, power source 404 may be capable of estimating the remaining time of operation using the current battery.

According to the techniques of this disclosure, external device 21 may be used to receive from PCD 110 and/or determine an indication of a measurement of hematocrit, an indication of true anemia, or an indication of fluid overload. For example, external device 21 may provide such indications to a clinician or other device to help guide treatment of patient 114.

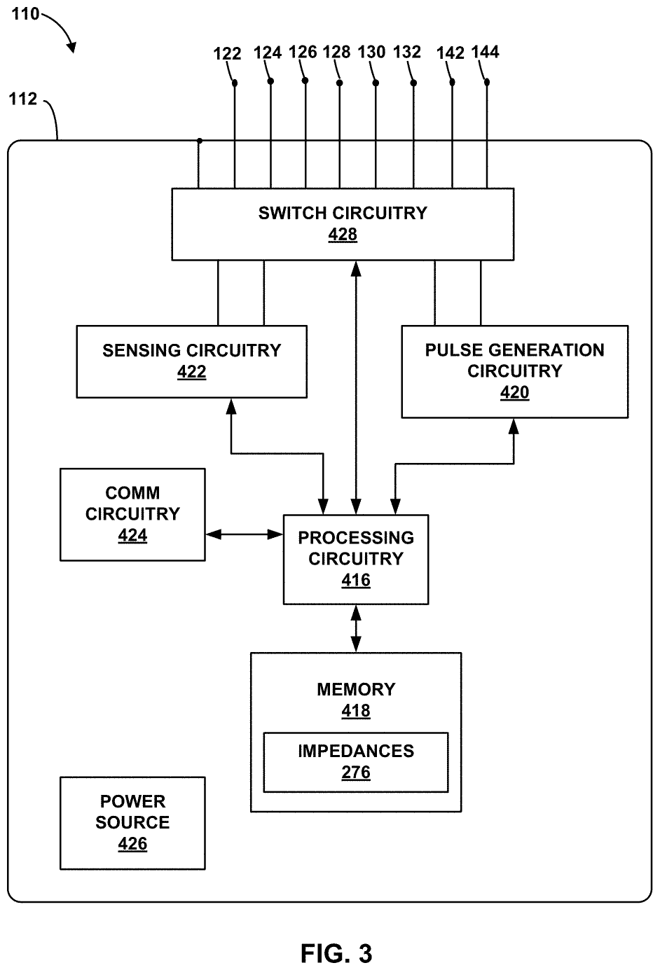

FIG. 3 is a functional block diagram illustrating an example configuration of pacemaker/cardioverter/defibrillator (PCD) 110. As illustrated in FIG. 3, in one example, PCD 110 includes sensing circuitry 422, pulse generation circuitry 420, processing circuitry 416 and associated memory 418, communication circuitry 424, power source 426, and switch circuitry 428. The electronic components may receive power from power source 426, which may be a rechargeable or non-rechargeable battery. In other examples, PCD 110 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitries may be performed by separate hardware or software components or integrated within common or separate hardware or software components.

Sensing circuitry 422 receives cardiac electrical signals from electrodes 112, 122, 124, 126, 128, 130, 132, 142 and 144 carried by right ventricular lead 118, left ventricular lead 120, and atrial lead 121, along with housing electrode 112 associated with the housing 112, for sensing cardiac events attendant to the depolarization of myocardial tissue, e.g., P-waves and R-waves, and sensing impedances indicative of hematocrit, true anemia, and/or fluid overload. In some examples, as illustrated in FIG. 3, PCD 110 may include switch circuitry 428 for selectively coupling electrodes 122, 124, 126, 128, 130, 132, 142, 144, and housing electrode 112 to sensing circuitry 422. Switch circuitry 428 may include a switch array, switch matrix, multiplexer, or any other type of switching device suitable to selectively couple one or more of the electrodes to sensing circuitry 422. In some examples, processing circuitry 416 selects the electrodes to function as sense electrodes, or the sensing vector, via switch circuitry 428. In some examples, PCD 110 may not include switch circuitry 428.

According to the techniques of this disclosure, processing circuitry 416 may determine a first impedance associated with a heart of patient 114 and determine a second impedance associated with the heart of patient 114. Processing circuitry 416 may determine a measure of hematocrit, an indication of fluid overload, or an indication of true anemia based at least in part on the first impedance and the second impedance. Processing circuitry 416 may output an indication of the measure of hematocrit, the indication of fluid overload, or the indication true anemia, for example, to external device 21 via communication circuitry 242, to inform a clinician.

Sensing circuitry 422 may include multiple sensing channels, each of which may be selectively coupled to respective combinations of electrodes 122, 124, 126, 128, 130, 132, 142, 144 and housing 112 to detect electrical activity of a particular chamber of heart 116, e.g., an atrial sensing channel and a ventricular sensing channel. Each sensing channel may comprise a sense amplifier that outputs an indication to processing circuitry 416 in response to sensing of a cardiac depolarization, in the respective chamber of heart 116. In this manner, processing circuitry 416 may receive sense event signals corresponding to the occurrence of sensed R-waves and P-waves in the respective chambers of heart 116. Sensing circuitry 422 may further include digital signal processing circuitry for providing processing circuitry 416 with digitized electrogram signals, which may be used for cardiac rhythm discrimination.

The components of sensing circuitry 422 may be analog components, digital components or a combination thereof. Sensing circuitry 422 may, for example, include one or more sense amplifiers, filters, rectifiers, threshold detectors, analog-to-digital converters (ADCs) or the like. Sensing circuitry 422 may convert the sensed signals to digital form and provide the digital signals to processing circuitry 416 for processing or analysis. For example, sensing circuitry 422 may amplify signals from the sensing electrodes and convert the amplified signals to multi-bit digital signals by an ADC. Sensing circuitry 422 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 416. In addition to detecting and identifying specific types of cardiac rhythms, sensing circuitry 422 may also sample the detected intrinsic signals to generate an electrogram or other time-based indication of cardiac events.

Processing circuitry 416 may process the signals from sensing circuitry 422 to monitor electrical activity of the heart of the patient. Processing circuitry 416 may store signals obtained by sensing circuitry 422 as well as any generated electrogram waveforms, marker channel data or other data derived based on the sensed signals in memory 418. Processing circuitry 416 may analyze the electrogram waveforms and/or marker channel data to detect cardiac events (e.g., tachycardia). In response to detecting the cardiac event, processing circuitry 416 may control pulse generation circuitry 420 to deliver the desired therapy to treat the cardiac event, e.g., ATP therapy.

In examples in which PCD 110 includes more than two electrodes for therapy delivery, processing circuitry 416 may use switch circuitry 428 to select which of the available electrodes are used to deliver pacing pulses. In some instances, the same switch circuitry may be used by both pulse generation circuitry 420 and sensing circuitry 422. In other instances, each of sensing circuitry 422 and pulse generation circuitry 420 may have separate switch circuitry.

Memory 418 may include computer-readable instructions that, when executed by processing circuitry 416, cause PCD 110 to perform various functions attributed throughout this disclosure to PCD 110 and processing circuitry 416. The computer-readable instructions may be encoded within memory 418. Memory 418 may comprise computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 416 may include any one or more of a microprocessor, a controller, a digital signal processor (DSP), an application specific integrated circuit (ASIC), a field-programmable gate array (FPGA), or equivalent discrete or integrated logic circuitry or state machine. In some examples, processing circuitry 416 may include multiple components, such as any combination of one or more microprocessors, one or more controllers, one or more DSPs, one or more ASICs, or one or more FPGAs, as well as other discrete or integrated logic circuitry or state machines. The functions attributed to processing circuitry 416 herein may be embodied as software, firmware, hardware or any combination thereof.

Pulse generation circuitry 420 is electrically coupled to electrodes 122, 124, 126, 128, 130, 132, 142, 144, and 112. In the illustrated example, pulse generation circuitry 420 is configured to generate and deliver electrical therapy to a heart of a patient. For example, pulse generation circuitry 420 may deliver the electrical therapy to a portion of cardiac muscle within the heart via any combination of electrodes 122, 124, 126, 128, 130, 132, 142, 144, and 112. In some examples, pulse generation circuitry 420 may deliver pacing stimulation, e.g., bradycardia therapy, CRT, or ATP therapy, in the form of voltage or current electrical pulses. In other examples, pulse generation circuitry 420 may deliver stimulation in the form of other signals, such as sine waves, square waves, or other substantially continuous time signals.

In some examples, in addition to cardiac pacing, PCD 110 may deliver cardioversion or defibrillation pulses. Pulse generation circuitry 420 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, pulse generation circuitry 420 may include a first set of components configured to provide pacing therapy and a second set of components configured to provide defibrillation therapy. In other instances, pulse generation circuitry 420 may utilize the same set of components to provide both pacing and defibrillation therapy. In still other instances, pulse generation circuitry 420 may share some of the defibrillation and pacing therapy components while using other components solely for defibrillation or pacing.

Processing circuitry 416 may control pulse generation circuitry 420 to deliver electrical therapy to heart 116 according to therapy parameters, which may be stored in memory 418. Pulse generation circuitry 420 is configured to generate and deliver electrical therapy to heart 116 via selected combinations of electrodes 122, 124, 126, 128, 130, 132, 142, 144, and housing electrode 112. For example, pulse generation circuitry 420 may be configured to deliver cardiac pacing via selected combinations of electrodes 122, 124, 126, 128, 130, and housing electrode 112, and may be configured to deliver antitachyarrhythmia shocks via selected combinations of electrodes 132, 142, 144, and 112.

As discussed above, processing circuitry 416 may determine a first impedance associated with a heart of patient 114 and determine a second impedance associated with the heart of patient 114 to determine a hematocrit value of patient 114 according to the techniques of this disclosure. In order to determine impedances, processing circuitry 416 may control pulse generation circuitry 420 to generate measurement or input signals (voltage or current) and sensing circuitry 422 may sense or measure resulting or output signals (current or voltage) via selected combinations of electrodes 122, 124, 126, 128, 130, 132, 142, 144, and housing electrode 112. Processing circuitry 416 may calculate or determine the impedances based on the memory 418 stores determined impedances 276, such as a first determined impedance and a second determined impedance. Such impedances may be used by processing circuitry 416 to determine a measure of hematocrit, an indication of true anemia, and/or an indication of fluid overload. Memory 418 may be further configured to store other sensed and detected data, therapy parameters, and any other information related to the therapy and treatment of a patient. In the example of FIG. 3, memory 418 may store sensed ECGs, detected arrhythmias, communications from PCD 100. In other examples, memory 418 may act as a temporary buffer for storing data until it can be uploaded to another implanted device, or external device 21.

Communication circuitry 424 is used to communicate with external device 21 for transmitting data accumulated by PCD 110 and for receiving interrogation and programming commands to and/or from external device 21. Communication circuitry 268 includes any suitable hardware, firmware, software or any combination thereof for communicating with another device, such as external device 21 (FIGS. 1 and 2), a clinician programmer, a patient monitoring device, or the like. For example, communication circuitry 424 may include appropriate modulation, demodulation, frequency conversion, filtering, and amplifier components for transmission and reception of data. Under the control of processing circuitry 416, communication circuitry 424 may receive downlink telemetry from and send uplink telemetry to external device 21 with the aid of an antenna, which may be internal and/or external. Processing circuitry 416 may provide the data to be uplinked to external device 21 and the control signals for the telemetry circuit within communication circuitry 424, e.g., via an address/data bus. In some examples, communication circuitry 424 may provide received data to processing circuitry 416 via a multiplexer.

In some examples, PCD 110 may signal external device 21 to further communicate with and pass the alert through a network such as the Medtronic CareLink® Network developed by Medtronic plc, of Dublin, Ireland, or some other network linking a patient to a clinician. PCD 110 may spontaneously transmit information to the network or in response to an interrogation request from a user.

Power source 426 may be any type of device that is configured to hold a charge to operate the circuitry of PCD 110. Power source 426 may be provided as a rechargeable or non-rechargeable battery. In other example, power source 426 may incorporate an energy scavenging system that stores electrical energy from movement of PCD 110 within patient 114.

The various circuitry of PCD 110 may include any one or more processors, controllers, digital signal processors (DSPs), application specific integrated circuits (ASICs), field programmable gate arrays (FPGAs), or equivalent discrete or integrated circuitry, including analog circuitry, digital circuitry, or logic circuitry.

PCD 110 may determine an impedance at two (or more) frequencies to determine a hematocrit level in patient 114. Impedance-based hematocrit determinations may rely on an inverse relation between hematocrit and the conductivity of blood σ_{b}. For example, σ_{b}∝1/HCT (hematocrit). However, when sensing hematocrit in-vivo, a possible parallel impedance (e.g., current through surrounding non-blood tissue) may affect a determination of a hematocrit level. This parallel admittance may occur because electrodes used to determine the impedance may be close to non-blood tissue. For example, the electrodes used to determine the impedance may be on right ventricular lead 118 or left ventricular lead 120.

In general, the measured complex admittance (=1/impedance) Y can be written as Y=G_{b}+Gₚ+j2πfCₚ, where G_{b}=blood conductance (=1/σ_{b}), and Yₚ(=Gₚ+j2πf Cₚ) is the complex parallel admittance

The parallel admittance includes a conductance Gp and a capacitance Cp, which are determined by the conductivity (σₚ) and the permissivity (εₚ) of the surrounding tissue, e.g. cardiac tissue.

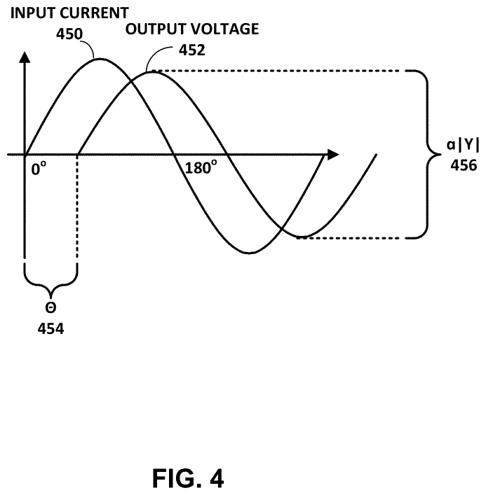

FIG. 4 is a graphical diagram illustrating an example phase shift between input current and an output voltage. In order to account for the parallel admittance, processing circuitry 416 of PCD 110 may determine a phase shift θ 454 between an input current 450 (e.g., a current signal delivered via a combination of electrodes) and an output voltage 452 (e.g., the voltage signal sensed via the combination of electrodes or another combination of electrodes) to determine a real (Re{Y}) and imaginary admittance (Im{Y}).

By determining a complex admittance at two (or more) different frequencies (f₁, f₂), e.g., using two (or more) input signals having different frequencies, G_{b} (blood conductance) can be determined, by exploiting the fact that σ_{b} is constant whereas σₚ and εₚ vary as a function of frequency.

For example, G_{b} may be determined by combining following formulas:

$${G}_{b} = Re \{ Y ( {f}_{1} ) \} - ( \frac{{σ}_{p , 1}}{{ε}_{p , 1}} ) \frac{Im \{ Y ( {f}_{1} ) \}}{2 π {f}_{1}}$$

$${G}_{b} = Re \{ Y ( {f}_{2} ) \} - ( \frac{{σ}_{p , 2}}{{ε}_{p , 2}} ) \frac{Im \{ Y ( {f}_{2} ) \}}{2 π {f}_{2}}$$

where the subscripts 1 and 2 refer to 2 different frequencies used.

If the ratio (σₚ/εₚ) is known (at either frequency), G_{b} can be calculated using one of the above equations. This is, however, typically not the case as these tissue properties differ across patients and may change over time. Using a common value across patients (e.g., retrieved from literature) for the ratio (σₚ/εₚ) may provide an approximate value for G_{b}.

To determine G_{b} more accurately, additional information about the relation between the ratio (σₚ/εₚ) at the different frequencies may be used (because the frequency dependence is more stable across patients, and over time). For example, if the ratio at the 2^{nd} frequency (σ_{p,2}/ε_{p,1}) can be written as K×(σ_{p,1}/ε_{p,1}), G_{b} may be determined using the following formula:

$${G}_{b} = \frac{Re \{ Y ( {f}_{1} ) \} - Re \{ Y ( {f}_{2} ) \} \frac{1}{K} ( \frac{{f}_{2}}{{f}_{1}} ) \frac{Im \{ Y ( {f}_{1} ) \}}{Im \{ Y ( {f}_{2} ) \}}}{1 - \frac{1}{K} ( \frac{{f}_{2}}{{f}_{1}} ) \frac{Im \{ Y ( {f}_{1} ) \}}{Im \{ Y ( {f}_{2} ) \}}}$$

The factor K may be determined from literature, or independent measurements of the electrical properties (σ, ε) of (cardiac) tissue.

An alternative embodiment uses information from a second set of impedance measurements in the same tissue but at a different condition (providing different admittance measurements). For example, such second set of data can be obtained from measurements at different states of contraction of the heart (e.g., systole vs diastole). In this case, for example, G_{b} may be determined using the following formula:

$${G}_{b} = \frac{Re \{ Y ( {f}_{2} ) \} - ( \frac{Im \{ Y ( {f}_{2} ) \}}{Im \{ {Y}_{c} ( {f}_{2} ) \}} ) [ Re \{ {Y}_{c} ( {f}_{1} ) \} - \\ Re \{ {Y}_{c} ( {f}_{2} ) \} - \frac{Im \{ {Y}_{c} ( {f}_{1} ) \}}{Im \{ Y ( {f}_{1} ) \}} Re \{ {Y}_{c} ( {f}_{1} ) \} ]}{1 - \frac{Im \{ Y ( {f}_{2} ) \} Im \{ {Y}_{c} ( {f}_{1} ) \}}{Im \{ Y ( {f}_{1} ) \} Im \{ {Y}_{c} ( {f}_{2} ) \}}}$$

where Y_{c} represents the admittance measured in the alternative condition.

Any of the above formulas may provide a quantity which is solely dependent on the conductivity of the blood and is not affected by the surrounding tissue. As the hematocrit is inversely related to the conductivity of the blood, the quantity, as such, can be used to monitor (relative) changes in hematocrit (independent from changes in the surrounding tissue).

In order to obtain an absolute value of the hematocrit, a calibration measurement can be performed during which the hematocrit is determined by an independent measurement technique (e.g., blood sampling).

$$HCT = \frac{{G}_{b}}{{G}_{b , c}} {HCT}_{c}$$

where G_{b,c} represents the (calculated) blood conductance (using any of the formulas above) from the calibration data, and HCT_{c} represents the hematocrit value at the time of calibration as determined by an independent hematocrit measurement technique.

In some examples, possible changes in volume and/or geometry may: 1) change the ratio G_{b}/σ_{b} and therefore G_{b}/hematocrit or 2) simultaneously change parallel admittance, which can be estimated by determining admittance (Y) at the two (or more) frequencies f₁ and f₂. Therefore, calibration using a known hematocrit level may be desirable to take account this effect. In some examples, processing circuitry 416 may determine a first measure of hematocrit based on the blood conductance (e.g., there may be an inverse relationship between hematocrit and blood conductance). In some examples, processing circuitry 416 may receive a second measure of hematocrit from another device (such as a hematocrit sensor), and based on the second measure, determine a calibration factor for processing circuitry 416 to apply to the first measure.

For example, the determination of hematocrit may be calibrated using the following formula:

$$HCT = \frac{{G}_{b}}{{G}_{b , c}} \frac{Δ {G}_{p}}{Δ {G}_{p , c}} \frac{Δ {G}_{p , c}}{Δ {G}_{p , {c}^{′}}} {HCT}_{c}$$

where HCT is the hematocrit level, subscripts c and c′ refer to calibration points, and ΔGₓ=Gₓ(f₂)−Gₓ(f₁).

For example, processing circuitry 416 may determine an impedance between an electrode on right ventricular lead 118 and housing electrode 112 and determine fluid in the thorax based on the determined impedance. In some examples, rather than using housing electrode 112, processing circuitry 416 may use an electrode on a lead, such as right ventricular lead 118, that is near a proximal end of the lead. Part of the current being used to stimulate presumably goes to lungs (fluid and lungs) of patient 114. Processing circuitry 416 may use a complex part of impedance to assess the effect that surrounding tissue has on the determined impedance. For example, if sensing circuitry 422 were to determine an impedance in the RV, part of current may travel to tissue which may make a determined value of impedance inaccurate. For example, blood may only be resistive, whereas tissue may be both resistive and conductive. By using complex impedances, PCD 110 may take into account the properties of surrounding tissue.

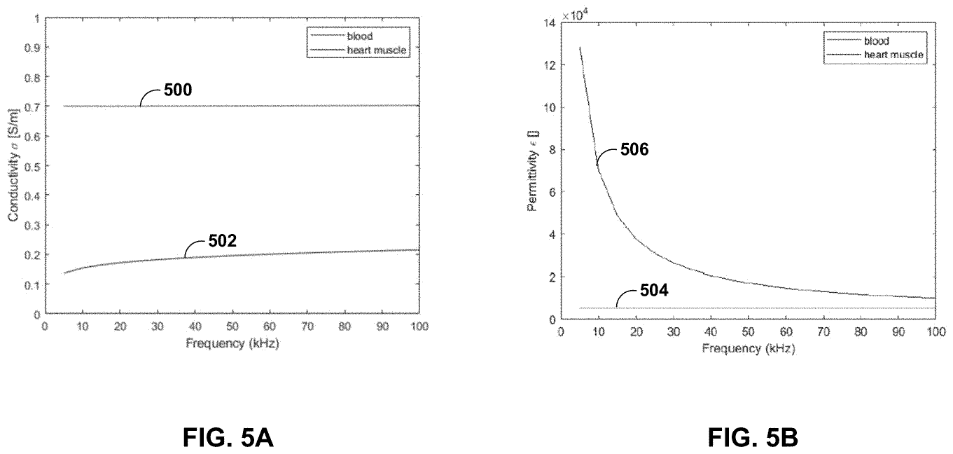

FIG. 5A is a graphical diagram illustrating an example of conductivity of heart and blood tissue as a function of frequency. As can be seen in FIG. 5A, the conductivity (Y-axis) of blood, shown as line 500, does not vary with frequency (X-axis). However, the conductivity of the heart muscle, shown as line 502, does vary with frequency.

FIG. 5B is a graphical diagram illustrating an example of permittivity of heart and blood tissue as a function of frequency. As can be seen in FIG. 5B, the permittivity (Y-axis) of blood, shown as line 504, does not vary with frequency (X-axis). However, the permittivity of the heart muscle, shown as line 506, does vary with frequency. At lower frequencies, the permittivity of blood is much smaller than that of the heart muscle.

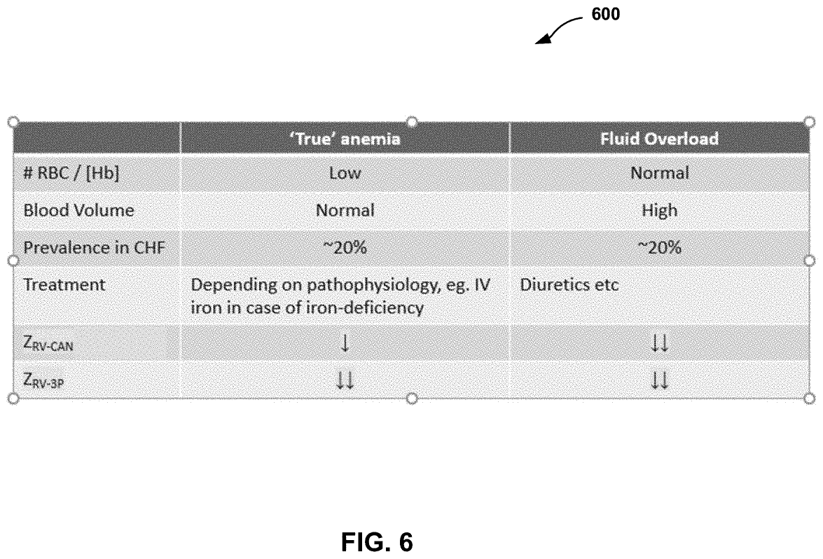

FIG. 6 is a chart illustrating an example relationship between impedance measurements which may be used to discriminate between true anemia and fluid overload according to the techniques of this disclosure. As discussed above, true anemia and fluid overload require different treatments. For example, treatment for true anemia may depend on pathophysiology, for example, intravenous administration of iron in the case of iron deficiency. Treatment for fluid overload may include, for example, the administration of diuretics to the patient. Therefore, it may be desirable to be able to distinguish or discriminate between true anemia and fluid overload and to output an indication of which condition patient 114 may have in order to guide a clinician in administering the appropriate treatment.

A determined hematocrit may be used to determine fluid status of patient 114 by taking into account the effect of the blood (hematocrit) on the intrathoracic impedance. In some examples, sensing circuitry 422 may determine intrathoracic impedance between an electrode on right ventricular lead 118 and housing electrode 112 of PCD 110. The intrathoracic impedance may be influenced by both the tissue and blood.

Certain medical devices, such as PCD 110, may be configured to periodically determine intrathoracic impedance. Such devices may be used to monitor fluid levels in patient 114 and may provide an early warning, for example, to a clinician via external device 21 for fluid-related decompensation. As, in some examples, the intrathoracic impedance is determined between an electrode on right ventricular lead 118 and housing electrode 112 of PCD 110. The intrathoracic impedance may be sensitive to fluid accumulation in the lungs (and interstitial tissue). Changes in other tissues (such as blood) between right ventricular lead 118 and PCD 110 can also influence these impedance measurements.

Processing circuitry 416 of PCD 110 may be configured to determine whether a condition is fluid overload and true anemia. For example, in case of fluid overload, both intrathoracic impedance and intracardiac impedance will decrease due to the decrease in hematocrit (e.g., hemodilution) and the fluid accumulation in the lung/interstitial tissue (between the right ventricle or right atrium and PCD 110). In case of “true” anemia, both impedances will also decrease but the effect on the intracardiac impedance will be larger than on the intrathoracic impedance because there is no change in the lung/interstitial fluid status. This difference can be seen in table 600 of FIG. 6. For example, sensing circuitry 422 may determine an intracardiac impedance and an intrathoracic impedance. Processing circuitry 416 may determine a relative change in intracardiac impedance compared to a baseline intracardiac impedance. Processing circuitry 416 may determine a relative change in intrathoracic impedance compared to a baseline intrathoracic impedance. Processing circuitry 416 may compare the relative change in intracardiac impedance to the relative change in intrathoracic impedance. Processing circuitry 416 may determine an indication of fluid overload or an indication of true anemia based on the comparison. For example, if the relative change in intracardiac impedance is greater than the relative change in intrathoracic impedance, processing circuitry 416 may determine an indication of true anemia. If the relative change in intracardiac impedance is less than or equal to the relative change in intrathoracic impedance, processing circuitry 416 may determine an indication of fluid overload. In some examples, rather than determining a relative change in intracardiac impedance and intrathoracic impedance, processing circuitry 416 may determine an absolute change in intracardiac impedance and an absolute change in intrathoracic impedance and compare the absolute changes to determine an indication of fluid overload or true anemia. In some examples, other measures may be determined as appropriate to determine an indication of fluid overload or true anemia.

In some examples, processing circuitry 416 may determine an indication of true anemia in patient 114. For example, processing circuitry 416 may determine an intracardiac impedance between electrodes on a pacing and/or defibrillation lead in a right ventricle or atrium, e.g., right ventricular lead 118. Processing circuitry 416 may determine the intrathoracic impedance between an electrode on a pacing and/or defibrillation lead in a right ventricle or atrium and housing electrode 112. Processing circuitry 416 may calculate the change in intracardiac and intrathoracic impedance compared to a baseline state of patient 114, the baseline state being a state where patient 114 has a normal hematocrit and fluid status. Processing circuitry 416 may compare the changes in intracardiac and intrathoracic impedance to determine whether the decrease in impedance is due to true anemia or fluid overload. In some examples, processing circuitry 416 may be configured to determine the condition of true anemia or fluid overload and discriminate therebetween even without quantifying an actual value of intracardiac impedance and an actual value of intrathoracic impedance.

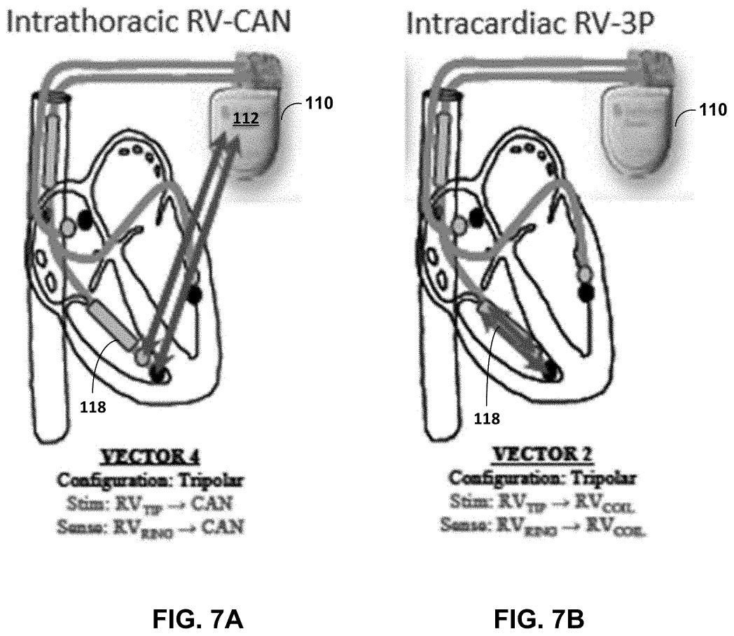

FIG. 7A is a conceptual diagram illustrating an example positioning of leads and electrodes used to determine an intrathoracic impedance according to the techniques of this disclosure. As shown in the example of FIG. 7A, intrathoracic impedance may be determined by stimulating (e.g., delivering an input signal) from a first electrode (e.g., tip electrode 122) of right ventricular lead 118 to housing electrode 112 of PCD 110 and sensing (e.g., sensing an output signal) between a second electrode (e.g., ring electrode 124) of right ventricular lead 118 and housing electrode 112 of PCD 110.

FIG. 7B is a conceptual diagram illustrating an example positioning of leads and electrodes used to determine an intracardiac impedance according to the techniques of this disclosure. As shown in the example of FIG. 7B, intracardiac impedance may be determined by stimulating from a first electrode (e.g., tip electrode 122) of right ventricular lead 118 to a second electrode (e.g., coil electrode 142) of right ventricular lead 118 and sensing between a third electrode (e.g., ring electrode 124) of right ventricular lead 118 and the second electrode of right ventricular lead 118.

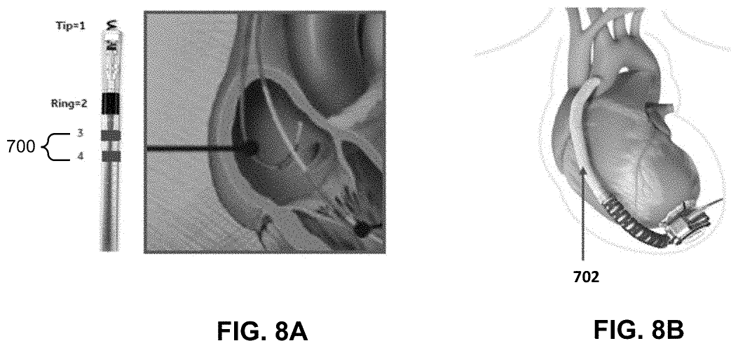

FIGS. 8A and 8B are conceptual diagrams illustrating example electrode locations. In some examples, if parallel admittance is negligible or not significant, an interelectrode distance 700 may be much smaller than a distance to tissue that is not blood. For example, Gp«G_{b}=>Re Y=|Y|=Gb∝σb. In some examples, the interelectrode distance 700 may be on the order of less than two millimeters, such as 1.2 mm.

In such a case, admittance magnitude |Y| may be measured at one frequency rather than two frequencies. An initial calibration may be performed, and the admittance may be used to determine a hematocrit level. For example, HCT=(|Yc|/|Y|)*HCTc where, Yc is the calibrated admittance and HCTc is the calibrated hematocrit level.

In the example of FIG. 8A, closely space electrodes 3 and 4 may be integrated into a bipolar lead to measure hematocrit in a heart chamber, such as a right atrium or right ventricle. In FIG. 8B, electrodes may be integrated into a ventricular assist device, such as a left ventricular assist device, in an outflow graft 702 or cannula to measure hematocrit.

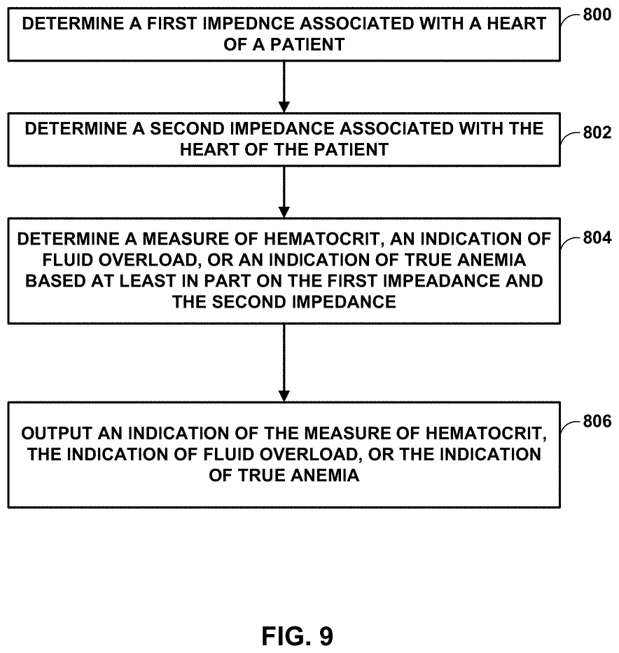

FIG. 9 is a flow diagram illustrating example hematocrit sensing techniques of this disclosure. The example technique of FIG. 8 is described as being performed by PCD 110, e.g., by sensing circuitry 422 and processing circuitry 416 of PCD 110. In some examples, the techniques may be performed in part by processing circuitry of another device, such as processing circuitry 400 of external device 21, based on data received from PCD 110. Furthermore, although described in the context of PCD 110, the techniques of this disclosure may be implemented in systems including other implantable or external devices configured measure impedances, such as other cardiac therapy and/or monitoring devices, ventricular assist devices (VADs), or neurostimulators.

PCD 110 may determine a first impedance associated with a heart of a patient (800). For example, PCD 110 may determine an impedance at a first frequency f₁. In another example, PCD 110 may determine an intracardiac impedance.

PCD 110 may determine a second impedance associated with the heart of the patient (802). For example, PCD 110 may determine an impedance at a second frequency f₂. In another example, PCD 110 may determine an intrathoracic impedance.

PCD 110 may determine a measure of hematocrit, an indication of fluid overload, or an indication of true anemia based at least in part on the first impedance and the second impedance (804). For example, PCD 110 may determine a measure of hematocrit using the determined impedance at the first frequency and the determined impedance at the second frequency. In another example, PCD 110 may determine an indication of fluid overload by determining that a relative change in intracardiac impedance is less than or equal to a relative change in intrathoracic impedance. In another example, PCD 110 may determine an indication of true anemia by determining that the relative change in intracardiac impedance is greater than the relative change in intrathoracic impedance.

PCD 110 may output an indication of the measure of hematocrit, the indication of fluid overload, or the indication true anemia (806). For example, PCD 110 may transmit to external device 21 the indication of the measure of hematocrit, the indication of fluid overload, or the indication true anemia. In this manner, a clinician may receive, through external device 21, the measure of hematocrit, the indication of fluid overload, or the indication of true anemia, and may determine an appropriate treatment for patient 114 based on the received indication.

In some examples, PCD 110 may determine a first complex impedance based on the first determined impedance, wherein the first determined impedance is determined at a first frequency; and determine a second complex impedance based on the second determined impedance, wherein the second determined impedance is determined at a second frequency. In some examples, determining the measure of hematocrit is based on the first complex impedance and the second complex impedance. In some examples, PCD 110 may determine a phase shift between an input current and an output voltage to calculate a real admittance and an imaginary admittance. In some examples, PCD 110 may determine blood conductance based on the first complex impedance and the second complex impedance, wherein the determining a measure of hematocrit is further based on the determined blood conductance. In some examples, PCD may calibrate the measure of hematocrit.

In some examples, the first impedance comprises an intracardiac impedance, the second impedance comprises an intrathoracic impedance, and the output comprises an indication of fluid overload or an indication of true anemia. In some examples, PCD 110 determines a relative change in intracardiac impedance compared to a baseline intracardiac impedance and determines a relative change in intrathoracic impedance compared to a baseline intrathoracic impedance. In some examples, PCD 110 compares the relative change in intracardiac impedance to the relative change in intrathoracic impedance, wherein the indication of fluid overload or the indication of true anemia is based on the comparison.

In some examples, as part of determining the intracardiac impedance, PCD 110 determines an impedance between electrodes in a ventricle or atrium. In some examples, as part of determining the intrathoracic impedance, PCD 110 determines an impedance between an electrode in a ventricle or atrium and a device housing electrode.

In some examples, the relative change in intracardiac impedance is greater than or equal to the relative change in intrathoracic impedance and wherein the indication comprises an indication of true anemia. In some examples, the relative change in intracardiac impedance is less than the relative change in intrathoracic impedance and wherein the indication comprises an indication of fluid overload.

Any suitable modifications may be made to the techniques described herein and any suitable device, processing circuitry, pulse generation circuitry, and/or electrodes may be used for performing the steps of the methods described herein. The steps of the methods may be performed by any suitable number of devices. For example, a processing circuitry of one device may perform some of the steps while a pulse generation circuitry and/or sensing circuitry of another device may perform other steps of the method, while communication circuitry may allow for communication needed for the processing circuitry to receive information from other devices. This coordination may be performed in any suitable manner according to particular needs.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to PCD 110, external device 21, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. For example, any of the techniques or processes described herein may be performed within one device or at least partially distributed amongst two or more devices, such as between PCD 110 and external device 21. In addition, any of the described units, circuitry or components may be implemented together or separately as discrete but interoperable logic devices. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media.

As used herein, the term “circuitry” refers to an application specific integrated circuit (ASIC), an electronic circuit, a processor (shared, dedicated, or group) and memory that execute one or more software or firmware programs, a combinational logic circuit, or other suitable components that provide the described functionality.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described for delivering cardiac stimulation therapies as well as coordinating the operation of various devices within a patient. Any combination of the described operations or functions is contemplated. These and other examples are within the scope of the following claims.

What is claimed is:

1. A method comprising:
determining, by processing circuitry, a first impedance associated with a heart of a patient;
determining, by the processing circuitry, a second impedance associated with the heart of the patient;
determining, by the processing circuitry, at least one of a measure of hematocrit, an indication of fluid overload, or an indication of true anemia based at least in part on the first impedance and the second impedance; and
outputting, by the processing circuitry, an indication of the measure of hematocrit, the indication of fluid overload, or the indication true anemia.

2. The method of claim 1, further comprising:
determining, by the processing circuitry, a first complex impedance based on the first determined impedance, wherein the first determined impedance is determined at a first frequency; and
determining, by the processing circuitry, a second complex impedance based on the second determined impedance, wherein the second determined impedance is determined at a second frequency,
wherein determining the measure of hematocrit comprises determining the measure of hematocrit based on the first complex impedance and the second complex impedance.

3. The method of claim 2, further comprising:
determining, by the processing circuitry, a phase shift between an input current and an output voltage to calculate a real admittance and an imaginary admittance.

4. The method of claim 2 or 3, further comprising:
determining, by the processing circuitry, blood conductance based on the first complex impedance and the second complex impedance, wherein the determining the measure of hematocrit is further based on the determined blood conductance.

5. The method of any of claims 2 to 4, further comprising:
calibrating, by the processing circuitry, the measure of hematocrit.

6. The method of claim 1, wherein the first impedance comprises an intracardiac impedance, the second impedance comprises an intrathoracic impedance, and the output comprises an indication of fluid overload or an indication of true anemia, further comprising:
determining, by the processing circuitry, a relative change in intracardiac impedance compared to a baseline intracardiac impedance;
determining, by the processing circuitry, a relative change in intrathoracic impedance compared to a baseline intrathoracic impedance; and
comparing, by the processing circuitry, the relative change in intracardiac impedance to the relative change in intrathoracic impedance,
wherein determining the indication of fluid overload or the indication of true anemia comprises determining the indication of fluid overload or the indication of true anemia based on the comparison.

7. The method of claim 6, wherein the determining the intracardiac impedance comprises determining the intracardiac impedance between electrodes in a ventricle or atrium.

8. The method of claim 6 or 7, wherein the determining the intrathoracic impedance comprises determining the intrathoracic impedance between an electrode in a ventricle or atrium and a device housing electrode.

9. The method of any of claims 6 to 8, wherein the relative change in intracardiac impedance is greater than or equal to the relative change in intrathoracic impedance and wherein the indication comprises an indication of true anemia.

10. The method of any of claims 6 to 8, wherein the relative change in intracardiac impedance is less than the relative change in intrathoracic impedance and wherein the indication comprises an indication of fluid overload.

11. A device comprising:
memory configured to store a first impedance and a second impedance, each associated with a heart of a patient; and
processing circuitry communicatively coupled to the memory and configured to:
determine at least one of a measure of hematocrit, an indication of fluid overload, or an indication of true anemia based at least in part on the first impedance and the second impedance; and
output an indication of the measure of hematocrit, the indication of fluid overload, or the indication true anemia.

12. The device of claim 11, wherein the processing circuitry is further configured to:
determine a first complex impedance based on the first determined impedance, wherein the first determined impedance is determined at a first frequency; and
determine a second complex impedance based on the second determined impedance, wherein the second determined impedance is determined at a second frequency,
wherein the determination of the measure of hematocrit is based on the first complex impedance and the second complex impedance.

13. The device of claim 12, wherein the processing circuitry is further configured to:
determine a phase shift in an input current and an output voltage to calculate a real admittance and an imaginary admittance.

14. The device of claim 12 or 13, wherein the processing circuitry is further configured to:
determine blood conductance based on the first complex impedance and the second complex impedance, wherein the determination of the measure of hematocrit is further based on the determined blood conductance.

15. The device of any of claims 12 to 14, wherein the processing circuitry is further configured to:
calibrate the measure of hematocrit.

16. The device of claim 11, wherein the first impedance comprises an intracardiac impedance, the second impedance comprises an intrathoracic impedance, and the output comprises an indication of fluid overload or an indication of true anemia, and wherein the processing circuitry is further configured to:
determine a relative change in intracardiac impedance compared to a baseline intracardiac impedance;
determine a relative change in intrathoracic impedance compared to a baseline intrathoracic impedance; and
compare the relative change in intracardiac impedance to the relative change in intrathoracic impedance,
wherein the indication of fluid overload or the indication of true anemia is based on the comparison.

17. The device of claim 16, wherein the processing circuitry is further configured to determine the intracardiac impedance between electrodes in a ventricle or atrium.

18. The device of claim 16 or 17, wherein the processing circuitry is further configured to determine the intrathoracic impedance between an electrode in a ventricle or atrium and a device housing electrode.

19. The device of claims 16 to 18, wherein the relative change in intracardiac impedance is greater than or equal to the relative change in intrathoracic impedance and wherein the indication comprises an indication of true anemia.

20. The device of claims 16 to 19, wherein the relative change in intracardiac impedance is less than the relative change in intrathoracic impedance and wherein the indication comprises an indication of fluid overload.

21. A non-transitory computer-readable medium including instructions, which, when executed, cause processing circuitry to:
determine at least one of a measure of hematocrit, an indication of fluid overload, or an indication of true anemia based at least in part on a first impedance and a second impedance associated with a heart of a patient; and
output an indication of the measure of hematocrit, the indication of fluid overload, or the indication true anemia.
